SEQUENCE LISTING (20 sequences)

SEQ ID NO: 1 - DNA, 333 bp, Homo sapiens
atgaggttcatgactctcctcttcctgacagctctggcaggagccctggtctgtgcctatgatccagaggccgcctctgccccaggatcggggaacccttgccatgaagcatcagcagctcaaaaggaaaatgcaggtgaagacccagggttagccagacaggcaccaaagccaaggaagcagagatccagccttctggaaaaaggcctagacggagcaaaaaaagctgtggggggactcggaaaactaggaaaagatgcagtcgaagatctagaaagcgtgggtaaaggagccgtccatgacgttaaagacgtccttgactcagtactatag

SEQ ID NO: 2 - DNA, 333 bp, Homo sapiens; identical to SEQ ID NO: 1

SEQ ID NO: 3 - DNA, 366 bp, Homo sapiens
atgaggttcatgactctcctcttcctgacagctctggcaggagccctggtctgtgcctatgatccagaggccgcctctgccccaggatcggggaacccttgccatgaagcatcagcagctcaaaaggaaaatgcaggtgaagacccagggttagccagacaggcaccaaagccaaggaagcagagatccagccttctggaaaaaggcctagacggagcaaaaaaagctgtggggggactcggaaaactaggaaaagatgcagtcgaagatctagaaagcgtgggtaaaggtggggaagagaggttggtctttggggctcctgtgaatctaacctccatccctctgacttctgtgagccgtccatga

SEQ ID NO: 4 - DNA, 57 bp, Homo sapiens
atgaggttcatgactctcctcttcctgacagctctggcaggagccctggtctgtgcc

SEQ ID NO: 5 - DNA, 90 bp, Homo sapiens
tatgatccagaggccgcctctgccccaggatcggggaacccttgccatgaagcatcagcagctcaaaaggaaaatgcaggtgaagaccca

SEQ ID NO: 6 - DNA, 276 bp, Homo sapiens
tatgatccagaggccgcctctgccccaggatcggggaacccttgccatgaagcatcagcagctcaaaaggaaaatgcaggtgaagacccagggttagccagacaggcaccaaagccaaggaagcagagatccagccttctggaaaaaggcctagacggagcaaaaaaagctgtggggggactcggaaaactaggaaaagatgcagtcgaagatctagaaagcgtgggtaaaggagccgtccatgacgttaaagacgtccttgactcagtactatag

SEQ ID NO: 7 - DNA, 33 bp, Homo sapiens
gaaaatgcaggtgaagacccagggttagccaga

SEQ ID NO: 8 - DNA, 87 bp, Homo sapiens
aaagctgtggggggactcggaaaactaggaaaagatgcagtcgaagatctagaaagcgtgggtaaaggagccgtccatgacgttaaa

SEQ ID NO: 9 - DNA, 138 bp, Homo sapiens
agccttctggaaaaaggcctagacggagcaaaaaaagctgtggggggactcggaaaactaggaaaagatgcagtcgaagatctagaaagcgtgggtaaaggagccgtccatgacgttaaagacgtccttgactcagta

SEQ ID NO: 10 - DNA, 141 bp, Homo sapiens
agccttctggaaaaaggcctagacggagcaaaaaaagctgtggggggactcggaaaactaggaaaagatgcagtcgaagatctagaaagcgtgggtaaaggagccgtccatgacgttaaagacgtccttgactcagtacta

SEQ ID NO: 11 - protein, 110 aa, Homo sapiens
MRFMTLLFLTALAGALVCAYDPEAASAPGSGNPCHEASAAQKENAGEDPGLARQAPKPRKQRSSLLEKGLDGAKKAVGGLGKLGKDAVEDLESVGKGAVHDVKDVLDSVL

SEQ ID NO: 12 - protein, 77 aa, Homo sapiens
MRFMTLLFLTALAGALVCAYDPEAASAPGSGNHKQMDCLQLQKPPSETAKFLSSSTNLPRREKLVPSAKPPHTRGLV

SEQ ID NO: 13 - protein, 121 aa, Homo sapiens
MRFMTLLFLTALAGALVCAYDPEAASAPGSGNPCHEASAAQKENAGEDPGLARQAPKPRKQRSSLLEKGLDGAKKAVGGLGKLGKDAVEDLESVGKGGEERLVFGAPVNLTSIPLTSVSRP

SEQ ID NO: 14 - protein, 19 aa, Homo sapiens
MRFMTLLFLTALAGALVCA

SEQ ID NO: 15 - protein, 30 aa, Homo sapiens
YDPEAASAPGSGNPCHEASAAQKENAGEDP

SEQ ID NO: 16 - protein, 91 aa, Homo sapiens
YDPEAASAPGSGNPCHEASAAQKENAGEDPGLARQAPKPRKQRSSLLEKGLDGAKKAVGGLGKLGKDAVEDLESVGKGAVHDVKDVLDSVL

SEQ ID NO: 17 - protein, 11 aa, Homo sapiens
ENAGEDPGLAR

SEQ ID NO: 18 - protein, 29 aa, Homo sapiens
KAVGGLGKLGKDAVEDLESVGKGAVHDVK

SEQ ID NO: 19 - protein, 47 aa, Homo sapiens
SSLLEKGLDGAKKAVGGLGKLGKDAVEDLESVGKGAVHDVKDVLDSV

SEQ ID NO: 20 - protein, 48 aa, Homo sapiens
SSLLEKGLDGAKKAVGGLGKLGKDAVEDLESVGKGAVHDVKDVLDSVL

(19) United States
(12) Patent Application Publication
(10) Pub. No.: US 2016/0361384 A1
(43) Pub. Date: Dec. 15, 2016

(54) COSMETIC USE OF DERMICIDIN, OR ANALOGUES OR FRAGMENTS THEREOF, FOR THE PREVENTION AND/OR TREATMENT AND DIAGNOSIS OF OILY SKIN AND AESTHETIC SKIN DISORDERS ASSOCIATED THEREWITH

(71) Applicant: L'OREAL, Paris (FR)

(72) Inventors: Audrey Gueniche, Rueil-Malmaison (FR); Dominique Bernard, Vanves (FR); Mark Donovan, Berville (FR)

(21) Appl. No.: 14/897,328

(22) PCT Filed: Jun. 16, 2014
(86) PCT No.: PCT/FR2014/051479

§ 371 Date: May 31, 2016

(30) Foreign Application Priority Data
Jun. 14, 2013 (FR) 1355577
Jun. 14, 2013 (FR) 1355579

(51) Int. Cl. A61K 38/17; G01N 33/94; C07K 14/47; G01N 33/68; A61Q 19/00; A61K 8/64

(52) U.S. Cl. CPC A61K 38/1709; A61Q 19/008; A61K 8/64; C07K 14/47; G01N 33/6881; G01N 33/9446; G01N 2800/20; G01N 2500/00

(57) ABSTRACT

Cosmetic or therapeutic, in particular dermatological, use of dermicidin, or analogues or fragments thereof, for preventing the appearance of imperfections, and also for the treatment and diagnosis of acneic skin or acne-prone skin. The subject matter of the present invention is in particular the use of a cosmetic and/or therapeutic, in particular dermatological, composition containing at least one amino acid sequence of dermicidin, or of an analogue or fragment thereof, or of at least one nucleic acid sequence encoding the sequence, for preventing the appearance of imperfections, and also for the treatment and/or prevention and/or diagnosis of acneic and/or oily or acne-prone and/or oily skin. It also related to a method for applying the abovementioned cosmetic composition for the prevention of the appearance of imperfections and the treatment of acne-prone and/or oily skin. It additionally relates to a method for treating acneic skin comprising the topical application to said skin of the pharmaceutical composition for the treatment of acneic and/or oily skin. The subject matter of the present invention is also the use of a biomarker for the diagnosis of acneic and/or oily or acne-prone and/or oily skin.

The present invention relates to the use of an amino acid sequence of dermcidin or of an analog or a fragment thereof, for preventing the appearance of imperfections or for the treatment and/or prevention and/or diagnosis of acneic skin or acne-prone skin, oily skin and/or esthetic skin disorders associated therewith.

It also relates to a cosmetic treatment process for the prevention and treatment of oily skin and/or esthetic skin disorders associated therewith.

The present invention also relates to the use of a biomarker for the diagnosis of acneic skin, oily skin or or skin which is acne-prone or prone to be oily.

Common acne is a multifactor disease (face, shoulder region, arms and intertriginous regions). It is the main cause of the most common forms of dermatosis. It is important not to trivialize this disease and to treat it correctly, since it can have debilitating psychosocial consequences, in particular because of the formation of scars.

In its mildest form, it affects almost every human being. Its frequency is maximal at the age of puberty, but it can manifest itself for the first time from the age of 7 to 9, and may extend beyond the age of 40. It is common to still be suffering from acne after the age of 25.

Acne is a disease of the sebaceous gland of the follicle. The following pathogenic factors play a determining role in the formation of acne:
Genetic predisposition.
Androgen.
Keratinization abnormally increased (keratinization disorder) at the level of the infundibular portion of the hair follicle. Indeed, in the deepest parts of the infundibulum, the formation of a larger than normal amount of keratinocytes is noted. These cells differentiate into horny cells which gradually block the lumen of the follicular canal. The physiological process of continuous desquamation from the acro-infundibulum to the surface is disrupted by the increased adhesion of the horny cells produced. A hyperkeratotic plug constituting the comedone, the initial lesion of acne, forms.
Bacterial infections responsible for the hydrolysis of free fatty acids and inflammatory phenomena (papules and pustules).

The predominant three local microorganisms, Staphyloccus epidermidis, Malassezia furfur and Propionibacterium acnes, find an ideal nutritive environment in the sebaceous follicle.

The clinical manifestations are characterized by a polymorphic picture. Interest is focused here on the most common forms of acne, namely, comedonal acne (juvenile acne), papulo-pustular acne and/or nodular acne.

The retention lesions may be of open or closed comedone type (microcyst, microcomedone, whitehead). The inflammatory lesions derived from the retention lesions may be of the type such as papules, pustules, with indurated nodules, abscesses, fistulas or scar forms.

### Evaluation Using the ECLA Scale

The clinical severity of acne has been determined using the semi-quantitative scoring scale ECLA [acne lesion clinical evaluation scale].

This scale is made up of three factors:

TABLE 1
Presentation of the ECLA scale.
Factor 1 (F1): type and intensity of the acne;
count performed on the entire face

| | | | | | | | Very | |
|---|---|---|---|---|---|---|---|---|
| | | Absent = | Rare = | Low = | Medium = | High = | high = | |
| | | 0 | 1 | 2 | 3 | 4 | 5 | F1 |
| R | Open and | None | <5 | 5 to 9 | 10 to 19 | 20 to 40 | >40 | R |
| | closed | | | | | | | |
| | comedones | | | | | | | |
| | (microcysts) | | | | | | | |
| Is | Papules | None | <5 | 5 to 9 | 10 to 19 | 20 to 40 | >40 | Is |
| | and pustules | | | | | | | |
| Ip | Inflam- | None | 1 | 2 | 3 | 4 | ≠5 | Ip |
| | matory | | | | | | | |
| | nodules | | | | | | | |
| | and cysts | | | | | | | |

Score 1 =

Factor 2 (F2): extension and intensity of the acne: beyond the face

| | | 0 | 1 | 2 | 3 | |
|---|---|---|---|---|---|---|
| | | Absent | Low | Medium | High | F2 |
| Neck (C) | Top | | | | | C |
| | cervical | | | | | |
| | zone | | | | | |
| | Bottom | | | | | |
| | cervical | | | | | |
| | zone | | | | | |
| Chest (P) | | | | | | P |
| Back (D) | Upper | | | | | D |
| | scapula tip | | | | | |
| | Lower | | | | | |
| | scapula tip | | | | | |
| Arm (B) | | | | | | B |
| Score 2 = | | | | | | |

Factor 3 (F3): scars absent = 0; present = 1

| Inflammatory | Non-inflammatory | Excoriations |
|---|---|---|
| CI | CNI | E |
| Score 3 = | | |

Final score: Score 1 + Score 2 + Score 3 =
The ECLA score is therefore between 0 and 36.

Hyperseborrhea or sebum overproduction is very common and results in skin with an oily, shiny appearance and enlarged pores. However, it is also very sensitive to environmental factors such as temperature, humidity or food. Moreover, it has been demonstrated that Propionibacterium acnes is directly involved in the stimulation of the IGF-1/IGF-1R axis in the skin, IGF-1R being a receptor which is found at the surface of the skin, and consequently induces hyperseborrhea and also proliferation and abnormal differentiation of keratinocytes (Isard et al., 2011). From a clinical point of view, studies demonstrate a direct correlation between the serum IGF-1 level and the amount of sebum secreted on the face (Aizawa and Niimura, 1995; Cappel et al., 2005; Vora et al., 2008). Furthermore, Linuma et at have been able to demonstrate, in vitro, an intracellular increase in the formation of lipid drops in sebocytes, an increase in triacylglycerol synthesis and also increase in sebum accumulation in the sebaceous glands and ducts after having exposed sebocyte cultures to a culture medium containing P. acnes strains. They have also confirmed these results in vivo by exposing hamster ears for 4 weeks to the P. acnes strain, demonstrating an increase in sebum accumulation in the sebaceous glands and ducts. The presence of P. acnes on the skin is therefore thought to be responsible for an overproduction of sebum and for oily skin and also skin disorders associated therewith.

Oily skin results most generally in shiny glistening skin and/or in skin imperfections such as dilated pores, a thick skin grain and/or a desquamation defect.

Consequently, hyperseborrhea due to the presence of P. acnes at the surface of the skin is clearly a phenomenon that appears to be important to effectively control in order to prevent the manifestation of associated esthetic skin disorders.

In order to combat pathogenic agents such as Propionibacterium acnes, active agents such as retinoids, azelaic acid, benzoyl peroxide, clindamycin, erythromycin and sodium sulfacetamide are commonly used. However, the use of these active agents is not without side effects. Thus, retinoids and azelaic acid are used only in the case of mild acne, since they are not very effective on inflammatory lesions and can cause dryness of the skin. Benzoyl peroxide is capable of causing skin irritations, whitening of the hair and also photoallergic reactions; it therefore has a not insignificant toxicity (Recherche chez l′homme du pouvoir phototoxique du peroxyde de henzoyle à 5% [Research in humans into the phototoxicity of benzoyl peroxide at 5%] M Jeanmougin, J Pedreiro, J Bouchet, J Civatte—Dermatology, 1983; Drug-induced photoallergic and phototoxic reactions. July 2007, Vol. 6, No. 4, Pages 431-443 Kevin R Stein & Noah S Scheinfeld). Clindamycin, erythromycin and sodium sulfamide in the form of salts are sensitizing substances capable of causing allergies, and leading to resistance (D Sasseville—Progrès en Dermato-Allergologie [Progress in Dermato-Allergology] Volume XVI: Strasbourg 2010).

There remains therefore the need for topically applied active agents which have an effect on microorganisms of the Propionibacterium genus, and particularly Propionibacterium acnes, while at the same time not having the drawbacks of the compounds known for this use.

Moreover, the development of resistance against some of these antimicrobials reveals the need to identify new molecules in order to maintain over time an effective action on the inhibition of P. acnes growth.

As it happens, the applicant has discovered that dermcidin, a natural antimicrobial, makes it possible to achieve this objective. This natural compound, unexpectedly, inhibits P. acnes growth.

In particular, the inventors have discovered that one of the peptides resulting from the cleavage of dermcidin, the antimicrobial peptide DCD-1L known to be present in sweat, is active on P. acnes growth and can therefore be used in a cosmetic composition, in particular as an active agent for preventing and/or treating oily skin and also esthetic skin disorders associated therewith.

A subject of the invention is therefore the use of a cosmetic composition containing at least one amino acid sequence of dermcidin, or of an analog or fragment thereof, for preventing and/or treating acneic skin and/or oily skin and esthetic skin disorders associated therewith.

The invention also relates to a cosmetic treatment process, in which a cosmetic composition comprising an amino acid sequence of dermcidin, or an analog or fragment thereof as defined hereinafter, is applied to the skin.

Another aspect of the invention is the use of dermcidin, or of an amino acid sequence derived from this protein, an analog or fragment thereof, or a nucleic acid sequence encoding an amino acid sequence in accordance with the invention, in particular as a biomarker for the diagnosis of oily skin.

For the purposes of the invention, the term “skin” is intended to denote the entire epidermis of the human body. More particularly, the skin considered in the present invention is preferably the skin of the face, of the neckline or of the back, and preferably the skin of the face.

For the purposes of the invention, the term “acne-prone skin” is intended to mean skin exhibiting acne with an ECLA score of less than 10, preferably of between 1 and 9.

For the purposes of the invention, the term “acneic skin” is intended to mean skin exhibiting acne with an ECLA score of between 10 inclusive and 36.

For the purposes of the invention, the term “oily skin” is intended to mean skin exhibiting hyperseborrhea characterized by an exaggerated secretion and excretion of sebum. Conventionally, a sebum level greater than 150/200 μg/cm² is considered to be characteristic of such oily skin.

For the purposes of the invention, the term “imperfections” is intended to mean temporary or permanent skin marks or scars due to acne lesions which are perceived by the subject to be unattractive.

For the purposes of the invention, the term “preventing” is intended to mean reducing the risk of occurrence or slowing down the occurrence of a given phenomenon, namely, in the present invention, skin marks and scars caused by spots, retention lesions of open or closed comedone type (microcyst, microcomedone, whitehead), inflammatory lesions derived from retention lesions of the type such as papules, pustules, with indurated nodules, abscesses, fistulas, scar forms, shiny glistening skin and/or skin with skin imperfections such as dilated pores, a thick skin grain and/or a desquamation defect.

The invention also relates to a cosmetic treatment process for the treatment of acne-prone skin and/or oily skin and for the prevention of imperfections, in which a cosmetic composition comprising an amino acid sequence of dermcidin, or an analog or fragment thereof as defined hereinafter, is applied to the skin.

The invention relates to a pharmaceutical, in particular dermatological, treatment process in which a pharmaceutical composition comprising dermcidin, an analog or fragment thereof or at least one amino acid sequence in accordance with the invention, at least one nucleic acid sequence encoding an amino acid sequence in accordance with the invention, or at least one agent for modulating the expression or the activity, which is in particular biological, of said amino acid sequence, is applied to the skin.

Another aspect of the invention is the use of dermcidin, or of an amino acid sequence derived from this protein, or an analog or fragment thereof, or a nucleic acid sequence encoding an amino acid sequence in accordance with the invention, in particular as a biomarker for the diagnosis of acneic or acne-prone skin or oily skin.

### Dermcidin

Dermcidin/Protease Inducing Factor (DCD/PIF) is a 110 amino acid (aa) protein comprising a 19aa signal peptide, the gene of which is located on chromosome 12, locus 12q13.1 and which is constitutively expressed in the sweat glands (Rieg, Garbe et al., 2004). Furthermore, two isoforms are listed in the UniProt base, a shorter one (isoform 1), and a longer one, isoform 2. It is secreted in sweat (Schittek, Hipfel et al., 2001). After proteolytic post-translational processing (essentially by cathepsin D), which takes place in sweat (Baechle, Flad et al., 2006), the protein precursor gives rise to numerous peptides, the length of which ranges from 25 to 48 aa (Flad, Bogumil et al., 2002). Some of these peptides, such as LL37 show antimicrobial activities against microorganisms of S. aureus, E. coli, Enterococcus faecalis, Candida albicans and P. acnes type. The antimicrobial peptides are derived from the C-terminal part of the protein. The activity of these peptides is independent of their charge (Steffen, Rieg et al. 2006) and is thought to involve a step of interaction with the bacterial phospholipids (Li, Rigby et al. 2009). The N-terminal part of the protein is constituted of a biologically active factor that promotes cell survival (Cunningham, Hodge et al., 1998). The decrease in DCD observed in the sweat of atopic skin types may partly explain the microbial susceptibilities of this skin type (Rieg, Steffen et al., 2005). A recent publication mentions the use of DCD as a particularly sensitive specific marker of the presence of traces of sweat by RT-PCR or by ELISA that can be used in the medico-legal field (Sakurada, Akutsu et al., 2009). It has been demonstrated that DCD links itself to bacterial phospholipids and self-organizes by forming ion channels in the bacterial membrane (Paulmann, Arnold et al. 2012).

Amino Acid and Nucleic Acid Sequences

Dermcidin (DCD) linked to its signal peptide is a protein of 110 amino acids (SEQ ID NO: 11) comprising a signal peptide of 19 amino acids (SEQ ID NO: 14), the gene of which is located on chromosome 12, locus 12q13.1. Two isoforms of this protein are listed, one being the shorter one, isoform 1 (SEQ ID NO: 12), the other being the longer one, isoform 2 (SEQ ID NO: 13).

After cleavage of the signal peptide, dermcidin (DCD) is a 90-amino acid protein having the sequence SEQ ID NO: 16.

After proteolytic post-translational maturation, essentially by cathepsin D, which takes place in sweat (Baechle, Flad et al., 2006), this protein precursor gives rise to DCD-1 (SEQ ID NO: 19) and to DCD-1L (SEQ ID NO: 20) and also to numerous peptides, the length of which ranges from 25 to 48 aa (Flad, Bogumil et al., 2002).

Unless otherwise indicated, the term “dermcidin” aims to denote in the present application the amino acid sequences represented by SEQ ID NO: 11, SEQ ID NO: 12, SEQ ID NO: 13, SEQ ID NO: 16, SEQ ID NO: 19 and SEQ ID NO: 20, optionally having undergone post-translational maturation.

According to one preferred embodiment, the term “dermcidin” is intended to denote the amino acid sequence represented by SEQ ID NO: 16.

According to an even more preferred embodiment, the term “dermcidin” is intended to denote more particularly the amino acid sequence represented by SEQ ID NO: 20.

The N-terminal part of the protein (SEQ ID NO: 15) is constituted of a biologically active factor that promotes cell survival (Cunningham, Hodge et al., 1998).

In accordance with the invention, the expression “analog of an amino acid sequence” is intended to denote any amino acid sequence having at least 85%, preferably at least 90%, more preferentially at least 95% and even more preferably at least 99% sequence identity with said sequence, and biological activity of the same nature.

The expression “biological activity of the same nature” with regard to an amino acid sequence according to the invention is intended to mean the antimicrobial properties or the properties of stimulation of cell survival and/or proliferation usually attributed to dermcidin.

The sequence identity may be determined by visual comparison or by means of any information technology tool generally used in the field, such as the Blast programs available on www.ncbi.nlm.nih.gov and used with the default parameters.

An analog in accordance with the invention may be a peptidomimetic agent.

An analog of an amino acid sequence of the invention may result from modifications derived from mutation or variation in the peptide sequences according to the invention originating either from the deletion or insertion of one or more amino acids, or from the substitution of one or more amino acids, or alternatively from alternative splicing. Several of these modifications may be combined.

Advantageously, an analog of an amino acid sequence of the invention may comprise conservative substitutions relative to this amino acid sequence.

As examples of mutations that may be considered in the present invention, mention may be made, in a non-exhaustive manner, of the replacement of one or more amino acid residues with amino acid residues having a similar hydropathic index, without, however, substantially affecting the biological properties of the polypeptide. The hydropathic index is an index assigned to amino acids according to their hydrophobicity and their charge (Kyte et al. (1982), J. Mol. Biol., 157: 105).

An amino acid sequence or an analog thereof targeted by the present invention may be an amino acid sequence that has undergone one or more post-translational maturation(s).

The term “post-translational maturation(s)” is intended to encompass all the modifications that an amino acid sequence is liable to undergo after its synthesis in a cell, for instance one or more phosphorylation(s), one or more thiolation(s), one or more acetylation(s), one or more glycosylation(s), one or more lipidation(s), such as a farnesylation or a palmitoylation, a structural rearrangement such as disulfide bridge formation and/or cleavage within the peptide sequence.

An analog of an amino acid sequence moreover has substantially the same biological activity as this amino acid sequence.

It is moreover known that a primary amino acid sequence may comprise sites specifically recognized by enzymes of protease type, such as trypsin, which, once the recognition of these sites is effective, will induce cleavage of the sequence by proteolysis. This proteolysis results in the generation of various peptides, or fragments of amino acid sequences of the invention.

Consequently, the invention also covers fragments of dermcidin, derived, where appropriate, from its proteolysis.

For the purposes of the invention, the term “fragment of an amino acid sequence” is intended to mean any portion of the amino acid sequence in accordance with the invention comprising from 3 to 48 consecutive amino acids of said sequence.

According to one embodiment, an amino acid sequence that is suitable for the invention may be an amino acid sequence represented by a sequence chosen from SEQ ID NO: 11 to 20, in particular from SEQ ID NO: 16, SEQ ID NO: 20, or an analog or fragment thereof.

According to one preferred embodiment, an amino acid sequence that is suitable for the invention may be an amino acid sequence represented by a sequence chosen from SEQ ID NO: 14, SEQ ID NO: 15, SEQ ID NO: 16, SEQ ID NO: 17, SEQ ID NO: 18, SEQ ID NO: 19, SEQ ID NO: 20, or an analog or fragment thereof.

More preferably, an amino acid sequence that is suitable for the invention may be an amino acid sequence represented by the sequence SEQ ID NO: 20, or an analog or fragment thereof.

According to another embodiment, a polypeptide that is suitable for the invention may also be a natural or synthetic amino acid sequence, where appropriate capable of being obtained by enzymatic or chemical hydrolysis of dermcidin or by chemical or biological synthesis or by extraction from a biological tissue, for instance the skin, expressing this amino acid sequence naturally or after transfection of said tissue, and also the various post-translational forms thereof, alternatively any natural or synthetic amino acid sequence of which the sequence totally or partially comprises an abovementioned amino acid sequence, for example the variants and the analogs.

Those skilled in the art can obtain an amino acid sequence in accordance with the invention by means of processes based on recombinant DNA, for instance those described in the manual “Molecular Cloning—A Laboratory Manual” (2nd edition), Sambrook et al., 1989, Vol. I-III, Coldspring Harbor Laboratory, Coldspring Harbor Press, NY, (Sambrook).

According to one embodiment, the present invention also relates to nucleic acid sequences SEQ ID NO:1 to SEQ ID NO:10 encoding respectively the amino acid sequences SEQ ID NO:11 to SEQ ID NO:20 of the invention, and the employment thereof in the various uses and processes in accordance with the invention.

For the purposes of the present invention, the expression “nucleic acid sequence fragment” is intended to mean a nucleic acid sequence encoding an amino acid sequence having biological activity of the same nature as the amino acid sequence encoded by said sequence.

For the purposes of the present invention, the expression “analog of a nucleic acid sequence” is intended to mean a nucleic acid sequence having at least 85%, preferably at least 90%, more preferentially at least 95% and even more preferably at least 99% sequence identity with said sequence, and encoding an amino acid sequence having biological activity of the same nature as the amino acid sequence encoded by said sequence.

The expression “analog of a nucleic acid sequence” is intended to denote a nucleic acid sequence optionally resulting from the degeneracy of the nucleic acid code, and encoding an amino acid sequence in accordance with the invention, in particular as previously defined.

According to one preferred embodiment, a nucleic acid sequence of the invention can be represented by at least one sequence chosen from SEQ ID NO: 6 or SEQ ID NO: 10 and more preferably is SEQ ID NO: 10.

A nucleic acid sequence of the invention may be of any possible origin, namely either animal, in particular mammalian and even more particularly human, or vegetable, or from microorganisms, for instance viruses, phages or bacteria inter alia, or alternatively from fungi, without any preconception regarding whether or not they are naturally present in said organism of origin.

According to one embodiment, the invention also relates to the isolated and purified nucleic acid sequences encoding an amino acid sequence under consideration according to the invention, and also to the analogs and fragments thereof.

A nucleic acid sequence in accordance with the invention may comprise a sense, antisense or interfering sequence corresponding to a sequence encoding a polypeptide in accordance with the invention.

Compositions

Cosmetic Composition

The present invention also relates to cosmetic compositions comprising in a cosmetically acceptable medium an effective amount of at least one amino acid sequence of the invention.

A composition of the invention may contain adjuvants that are usual in the field under consideration, such as hydrophilic or lipophilic gelling agents, preservatives, antioxidants, solvents, fragrances, fillers, screening agents, odor absorbers and colorants, oils, water waxes and polyols.

The amounts of the various constituents in the compositions according to the invention are those conventionally used in the fields under consideration.

The amount of amino acid sequence, or of nucleic acid sequence of the invention, or of active agent in accordance with the invention contained in a composition of the invention, also known as the “effective amount”, depends, of course, on the nature of the active agent and of the desired effect and may thus vary within a wide range.

To give an order of magnitude, a composition may contain an amino acid sequence, a nucleic acid sequence or an active agent in accordance with the invention, in an amount representing from 0.00001% to 2.5% of the total weight of the composition, in particular in an amount representing from 0.001% to 2% of the total weight of the composition and more particularly in an amount representing from 0.1% to 1% of the total weight of the composition.

For the purposes of the present invention, the term “effective amount” of a compound of the invention is intended to mean an amount of this compound which is sufficient and necessary to obtain a desired effect, and more particularly a cosmetic or care effect with regard to acneic skin. Thus, and without it being in any way limiting for the purposes of the present invention, those skilled in the art will be able to refer to the examples hereinafter in which said at least one amino acid sequence represents 0.01% of the composition.

Pharmaceutical Composition

The present invention also relates to pharmaceutical compositions, in particular dermatological compositions, comprising, in a physiologically acceptable medium, an effective amount of at least one amino acid sequence in accordance with the invention, or of at least one nucleic acid sequence in accordance with the invention, or of at least one active agent capable of modulating the activity, the expression or the maturation of said amino acid sequence or of said nucleic acid sequence.

For the purposes of the present invention, the term “modulating agent” or “active agent capable of modulating the expression, the maturation or the activity of an amino acid sequence or of a nucleic acid sequence in accordance with the invention” is intended to mean any compound capable of acting, directly or indirectly, on at least one amino acid sequence or one nucleic acid sequence in accordance with the invention, or on an element of an intracellular or extracellular signalling pathway, or of a metabolic pathway, or of a pathway for regulating transcription and/or translation which involves said amino acid sequence or said nucleic acid sequence.

For the purposes of the invention, the term “modulating”, with regard to a given effect, is intended to mean the action of stimulating or inhibiting this effect.

For the purposes of the present invention, the term “physiologically acceptable medium” is intended to denote a medium that is suitable for topical administration, to the skin, the scalp or the lips, or oral or parenteral administration, such as intradermal or subcutaneous administration, of a composition.

The amounts of the various constituents in the compositions according to the invention are those conventionally used in the fields under consideration.

The amount of amino acid sequence, or of nucleic acid sequence of the invention, or of active agent in accordance with the invention contained in a composition of the invention, also known as the “effective amount”, depends, of course, on the nature of the active agent and of the desired effect and may thus vary within a wide range.

To give an order of magnitude, a composition may contain an amino acid sequence, a nucleic acid sequence or an active agent in accordance with the invention, in an amount representing from 0.00001% to 5% of the total weight of the composition, in particular in an amount representing from 0.001% to 5% of the total weight of the composition, preferentially from 0.1% to 5% and more particularly in an amount representing from 0.5% to 5% of the total weight of the composition, and even more preferably from 2.5% to 5%.

According to another embodiment, a composition at least one additional therapeutic active agent.

Cosmetic Use

The present invention relates to the cosmetic use of an effective amount of at least one amino acid or nucleic acid sequence, or of as an active agent for preventing and/or treating acneic skin and/or oily skin and/or the appearance of esthetic skin disorders associated therewith.

According to another aspect, the present invention relates to a non-therapeutic cosmetic process for preventing and/or treating acne-prone skin and/or oily skin and/or the appearance of esthetic skin disorders associated therewith, in an individual who is in need thereof. The process comprises at least one step consisting in administering, to said individual, at least one composition comprising, as active agent, (i) at least one amino acid sequence of the invention, in particular as defined above.

A process or a use of the invention makes it possible to prevent and/or treat oily skin and/or the appearance of esthetic skin disorders associated therewith.

A process or a use of the invention makes it possible to confer and/or to return to healthy skin.

For the purposes of the invention, the term “healthy skin” is intended to mean non-shiny, non-glistening skin which exhibits few or no esthetic skin disorders such as dilated pores, comedones, blackheads or a thick microrelief.

Preferably, a process of the invention may comprise the topical application, to at least a part of the skin of an individual who is in need thereof, in particular to the skin of the face, the back, the neckline and/or the scalp, of at least one coat of a topical composition of the invention.

A cosmetic process according to the invention may be performed daily, for example at a rate of a single application per day or of an administration split into two or three times per day, for example once in the morning and once in the evening.

A cosmetic process according to the invention may be implemented over a time period ranging from one week to several weeks, or even several months, this period moreover possibly being repeated after periods without treatment, for several months or even several years.

By way of example of a cosmetic process according to the invention, it is possible to envision application of a cosmetic composition of the invention, for example, at a rate of 1, 2 or 3 times per day, or more, and generally over an extended period of at least 4 weeks, or even 4 to 15 weeks, with, where appropriate, one or more periods of stoppage.

In a second embodiment, the present invention relates to the field of biomarkers of the skin, and more particularly of oily, hyperseborrheic skin, and also to the use thereof as targets or cosmetic active agents.

These factors can be used as biomarkers of the skin, as screening targets, or even as cosmetic active agents.

There is also a need to have available novel biomarkers which make it possible to characterize a condition of the skin, and in particular of acne-prone skin, of oily skin or of skin with imperfections, in order to obtain quantitative data which make it possible to objectively evaluate this physiological condition.

Therapeutic Use

The present invention relates to the therapeutic use of an effective amount of at least one amino acid or nucleic acid sequence of the invention or of at least one agent for modulating the activity, the expression or the maturation of said amino acid sequence or of said nucleic acid sequence, and in particular a modulating agent as defined previously, as an active agent for preventing the appearance of imperfections and/or preventing and/or treating acneic skin.

According to another aspect, the present invention relates to a therapeutic process for preventing the appearance of imperfections and/or preventing and/or treating acneic skin in an individual who is in need thereof, the process comprising at least one step consisting in administering, to said individual, at least one composition comprising, as active agent, (i) at least one amino acid sequence of the invention, or (ii) at least one nucleic acid sequence of the invention, or (iii) at least one agent for modulating the activity, the expression or the maturation of said sequences of the invention, in particular as defined above.

A process or a use of the invention makes it possible to return to healthy skin, manifesting itself through skin which is smooth and without imperfections.

A process or a use of the invention makes it possible to prevent and/or reduce the presence of a microrelief of the skin.

Preferably, a process of the invention may comprise the topical application, to at least a part of the skin of an individual in need thereof, in particular to the skin of the face, the back and/or the neckline, of at least one coat of a topical composition of the invention.

A therapeutic process by topical application according to the invention may advantageously comprise the application of a composition of the invention, in combination, simultaneously, successively or separately over time, with an additional cosmetic or dermatological composition distinct from the composition of the invention and intended for skin care.

A pharmaceutical process according to the invention may be performed daily, for example at a rate of a single application per day or of an administration split into two or three times per day, for example once in the morning and once in the evening.

A pharmaceutical process according to the invention may be implemented over a time period ranging from one week to several weeks, or even several months, this period moreover possibly being repeated after periods without treatment, for several months or even several years.

By way of example of a pharmaceutical process according to the invention, it is possible to envision application of a composition of the invention, for example, at a rate of 1, 2 or 3 times per day, or more, and generally over an extended period of at least 4 weeks, or even 4 to 15 weeks, with, where appropriate, one or more periods of stoppage.

In a second embodiment, the present invention relates to the field of biomarkers of the skin, and more particularly of acneic skin, and also to the use thereof as targets or cosmetic active agents.

These factors can be used as biomarkers of the skin, as screening targets, or even as cosmetic active agents.

There is also a need to have available novel biomarkers which make it possible to characterize a condition of the skin, and in particular of acneic or acne-prone skin, or skin with imperfections.

Biomarker

To the inventors' knowledge, this antimicrobial protein of the skin surface has never been categorized as a variant of acneic and/or oily skin and even less so as one of the most relevant biomarkers emerging from studies of this type.

Unexpectedly, during a differential proteomic study using the “iTRAQ” technology, the inventors have observed, on the basis of proteins extracted from varnish strippings taken from 24 hyperseborrheic subjects and 24 subjects with normal seborrhea, of different age, that dermcidin (or DCD) proves to be a sensitive and specific biomarker for oily skin.

More specifically, the inventors have observed that the level of expression of dermcidin, and more particularly of peptides derived from dermcidin and identified by the sequences SEQ ID NO: 11 to SEQ ID NO: 20 and in particular SEQ ID NO: 20 are systematically decreased in oily skin compared with healthy skin, in particular with a ratio of on average 0.32 obtained with 3 independent experiments.

To the inventors' knowledge, this antimicrobial protein of the skin surface has never been categorized as a variant of oily skin and even less so as one of the most relevant biomarkers emerging from studies of this type.

It is known that sweating decreases with age (Anderson and Kenney 1987; Kenney and Fowler 1988) and that dermcidin is a sweat protein (Schittek, Hipfel et al., 2001). However, the experimental data obtained by the inventors show, unexpectedly, that there is a specific deficit in the expression and/or maturation of DCD in oily skin since said DCD is under expressed in the skin samples.

The present invention relates to the use of at least one amino acid sequence of the invention, or of at least one nucleic acid sequence of the invention, as a biomarker of a condition of the skin.

The present invention relates to the use of at least one amino acid sequence of the invention, or of at least one nucleic acid sequence of the invention, as a biomarker of a condition of the skin.

Preferably, a use in accordance with the invention makes it possible to characterize a condition of the skin such as acneic and/or oily skin.

The present invention relates to an in vitro or ex vivo process for characterizing an acneic and/or oily condition of the skin of a subject, comprising at least the steps consisting in:

a) performing, in an isolated sample of skin of a subject, a qualitative or quantitative measurement of the expression, maturation or activity of the biomarker, and

b) comparing said measurement performed in step a) with a reference measurement,

c) concluding that a reduction in the activity, expression or maturation of said biomarker relative to said reference measurement is indicative of acneic and/or oily or acne-prone and/or oily skin in the subject.

For the purposes of the invention, the term “biomarker” is intended to mean a molecule or the activity of a molecule of which the presence, content or degree of activity is characteristic of a biological, physiological or pathological process, or of the impact or effect induced by the administration of an active agent on such a process. Preferably, the use of dermcidin, or of an amino acid sequence derived from this protein, or of an analog or fragment thereof, or a nucleic acid sequence encoding an amino acid sequence in accordance with the invention, in particular as a biomarker for the evaluation of a condition of the epidermis, which may be one of the causes of the “abnormal” proliferation, such as P. acnes.

According to one embodiment, a decrease in the activity, expression or maturation of said biomarker may be indicative of acneic and/or oily or acne-prone and/or oily skin.

According to one embodiment, an increase in the activity, expression or maturation of said biomarker may be indicative of a cosmetic treatment which is effective for exerting a beneficial effect on the skin and more preferentially an effect with regard to acneic and/or oily or acne-prone and/or oily skin.

In a use in accordance with the invention, a decrease or an increase in the activity, expression or maturation of said biomarker may be determined by comparison with a reference measurement obtained according to any method known to those skilled in the art.

A “reference measurement” with regard to a given parameter is a qualitative or quantitative measurement of this parameter performed under “control” or “normal” conditions, for example determined in a reference sample, or determined in a sample in the absence of a treatment presumed to have an effect on the parameter.

For example, a reference measurement for an amino acid sequence or a nucleic acid sequence in accordance with the invention may be a quantitative or qualitative value relative to the expression, maturation or activity of said sequences determined in a sample of physiologically healthy skin, or determined in a sample of skin, in particular of acneic skin, before a cosmetic or therapeutic treatment.

Preferably, a reference measurement is a statistical measurement, i.e. a measurement that has been repeated on various samples so as to obtain an average.

The reference measurement may be taken in parallel with or sequentially to the test measurement.

It may also be a “historical” measurement, i.e. a measurement taken prior to the test measurement, and stored, for example, in a database, for the purpose of subsequent use.

A comparison of the test measurement with a reference measurement, and an observation of a deviation or an absence of deviation between the two measurements, makes it possible to draw information regarding the measured parameter, for example the decrease or increase in the expression, maturation or activity of an amino acid sequence or of a nucleic acid sequence in accordance with the invention.

Such information can be subsequently used to determine the healthy or acne-prone and/or acneic nature of a skin.

According to one embodiment, the invention relates to a process, in particular an in vitro or ex vivo process for characterizing a condition of acneic and/or oily or acne-prone and/or oily skin.

The qualitative or quantitative measurement of the expression, of the maturation or of the activity of a nucleic acid sequence of the invention may be determined using any method known to those skilled in the art.

By way of example of methods that are suitable for the invention, mention may be made of the polymerase chain reaction (PCR), which may be quantitative (Q-PCR) or non-quantitative, optionally in the presence of reverse transcriptase (RT-PCR or Q-RT-PCR), Northern blotting, the ribonuclease protection assay method, methods with DNA chips, methods with transcriptome chips, methods with oligonucleotide chips, or in situ hybridization methods.

By way of example of agents that are suitable for the detection of a nucleic acid sequence of the invention, and in particular of an mRNA sequence, mention may be made of labeled nucleic acid probes which can hybridize to a nucleic acid sequence of the invention.

Such a nucleic acid probe can be easily obtained by any method known to those skilled in the art.

Thus, the nucleic acid sequences in accordance with the invention may be used for producing sense and/or antisense oligonucleotide primers which hybridize under high stringency conditions to at least one of the sequences SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, or an analog or fragment thereof.

The expression of a nucleic acid sequence may also be determined, indirectly, by determining the expression of the amino acid sequence encoded by said sequence, by means of any technique known in the field, such as Western blotting, ELISA, the BRADFORD or LOWRY method, or as indicated hereinafter.

The qualitative or quantitative measurement of the expression, maturation or activity of an amino acid sequence of the invention may be performed using any method known to those skilled in the art.

By way of methods for detecting the expression, maturation or activity of an amino acid sequence, mention may be made of Western blotting, slot blotting, dot blotting, ELISA (Enzyme Linked Immuno-Sorbent Assay) methods of singleplex or multiplex type, proteomic or glycomic methods, methods for staining polypeptides in a polyacrylamide gel with a silver-based stain, with Coomassie blue or with SYPRO, immunofluorescence methods, UV absorption methods, immunohistochemical methods by conventional, electron or confocal microscopy, FRET (fluorescence resonance energy transfer) methods, TR-FRET (time resolved FRET) methods, FLIM (fluorescence lifetime imaging microscopy) methods, FSPIM (fluorescence spectral imaging microscopy) methods, FRAP (fluorescence recovery after photobleaching) methods, reporter gene methods, AFM (atomic force microscopy) methods, surface plasmon resonance methods, microcalorimetry methods, flow cytometry methods, biosensor methods, radioimmunoassay (RIA) methods, isoelectric focusing methods, and enzymatic tests, methods using peptide chips, sugar chips, antibody chips, mass spectrometry methods, and spectrometry methods of SELDI-TOF type (Ciphergen).

More generally, immunoenzymatic assay methods using protein solutions, which are more quantitative and sensitive, may in particular be used. These ELISA-type methods combine pairings of target-antigen-specific capture antibody and detection antibody. Commercial antibodies or specifically developed monoclonal, polyclonal or recombinant antibodies may be used. High-capacity multiplex ELISA techniques may also be used. Mention may thus be made of the multiplex approach of the type such as antibodies on Luminex beads (for example Bioplex from Bio-Rad) or of the type such as antibodies on a flat surface (antibody arrays) (for example the approach proposed by the company MesoScale Discovery). Mention may also be made of the microfluidic cartridge technology developed by the Korean company NanoEntek. In a recent approach, a novel technique based on “quench body” antibodies proposed by the Japanese company Ushio makes it possible to use only an antibody.

In particular, it may be advantageous to detect the expression of an amino acid sequence of the invention by means of an antibody, where appropriate in a labeled form. Such an antibody may be labeled with a directly detectable substance or a substance that is detectable by reaction with another reagent.

The term “antibody” is intended to denote, generally, monoclonal or polyclonal antibodies, and also immunoglobulin fragments capable of binding an antigen and which can be produced by any genetic engineering technique known to those skilled in the art or by enzymatic or chemical cleavage of an intact antibody.

An antibody that may be used as a tool for evaluating the condition of an epidermis may be obtained via any process known to those skilled in the art, as described in “Antibodies: A Laboratory Manual”, Cold Spring Harbor Laboratory Press, Cold Spring Harbor, N.Y. (1990).

According to one preferred embodiment, it may be advantageous to detect the expression of an amino acid sequence of the invention by means of an “iTRAQ” differential proteomic method.

Such a method is known to those skilled in the art and may be advantageously performed as described in the examples below.

In particular, the term “activity” with regard to an amino acid sequence of the invention is intended to mean an antimicrobial activity, a cell survival-stimulating activity, a cell proliferation-stimulating activity or an activity for reducing or preventing aged skin and/or signs of skin aging, as indicated above.

Such an activity can be determined by any method known to those skilled in the art, for instance by evaluating the proliferation or survival of epidermal cells in cultures, such as keratinocytes, or by evaluating the antimicrobial activity on bacteria in culture, such as Staphylococcus aureus, Escherichia coli or P. acnes.

Preferably, the determination of a condition of the skin or the characterization of the efficacy of a cosmetic treatment of the skin may be performed by measuring the variation in the expression of an amino acid sequence of the invention, and preferably represented by a sequence chosen from SEQ ID NO: 16, SEQ ID NO: 17, SEQ ID NO: 18, SEQ ID NO: 19 or SEQ ID NO: 20, or an analog or fragment thereof.

The processes of the invention are particularly advantageous since the implementation thereof does not require recourse to an invasive technique. A sample of epidermis may thus be obtained by “stripping” techniques and directly analyzed by a conventional analytical technique known to those skilled in the art.

These strippings are adhesive surfaces applied to the surface of the epidermis, such as Blenderm® from 3M, D′squam (commercial adhesive from CuDERM), cyanoacrylate adhesive or the varnish “stripping” method. By virtue of these “strippings”, the adherent corneocytes and the content of their intercellular spaces can be sampled and subsequently subjected to an extraction making it possible to have access to the protein content.

The taking of a sample that is suitable for a process of the invention can also be carried out more directly by “washing” the skin surface, by means for example of accessories of the vane turbine type, or of the spiral cell type as described in patent FR 2 667 778 associated with a fluidic circuit, or simply by adding/removing a drop of buffer at the surface of the skin.

By way of indication, other sampling methods suitable for implementing the invention may be mentioned, such as methods by scraping the upper part of the stratum corneum by means of a twin blade system. This technique makes it possible to collect squamae which can then be directly analyzed by various techniques in order to determine the mineral, amino acid or lipid contents.

Advantageously, one of the markers of the invention may be used for the purposes of more efficient and more rigorous preclinical selection, of individuals, with a view to evaluating the efficacy of a cosmetic treatment or active agent for skin care.

Similarly, a biomarker of the invention may advantageously be used as previously indicated for evaluating the efficacy of an active agent, in vitro, ex vivo or in vivo.

Similarly, a biomarker of the invention may be used for establishing personalized advice for a cosmetic treatment for an individual according to said individual's skin biomarker expression profile.

According to yet another of its subjects, the present invention relates to the use (i) of at least one amino acid sequence of the invention, or (ii) of a nucleic acid sequence of the invention for screening for active agents that are capable of modulating the activity, expression or maturation of said amino acid sequence or of said nucleic acid sequence.

For the purposes of the invention, the term “expression” with regard to an amino acid sequence, for example a protein or a peptide, or to a nucleic acid sequence, for example an mRNA, is intended to mean its content or the variation of its content relative to a reference.

For the purposes of the invention, the term “maturation” with regard to an amino acid sequence, for example a protein or a peptide, or to a nucleic acid sequence, for example an mRNA, is intended to mean the modifications that follow their synthesis in a cell environment. For example, in the case of an amino acid sequence, the term “maturation” is intended to mean the post-translational modifications, such as glycosylation or farnesylation of certain amino acids, or the proteolytic steps leading to the removal of “signal” or “secretory” sequences or to the release of sequences having particular biological properties. In the case of a nucleic acid sequence, the term “maturation” is intended to mean, for example, the alternative splicing of an mRNA.

For the purposes of the invention, the term “activity” is intended to mean, with regard to an amino acid sequence, for example a protein or a peptide, the biological activity of the amino acid sequence, where appropriate after maturation, such as an enzymatic activity, an agonist or antagonist activity with respect to a receptor, an enzyme-activating or -inhibiting activity, a “structural” activity, or an antimicrobial activity.

For the purposes of the invention, the term “activity” is intended to mean, with regard to a nucleic acid sequence, for example an mRNA, its translation.

According to another of its subjects, the present invention relates to the use (i) of at least one amino acid sequence of the invention, or (ii) of at least one nucleic acid sequence of the invention, for characterizing the efficacy of a treatment of acneic and/or oily or acne-prone and/or oily skin.

Likewise, according to another advantage, the present invention makes it possible to propose novel active agents that are suitable for the prevention and/or treatment of acneic and/or oily or acne-prone and/or oily skin.

Screening

There is still a need to have available novel tools for screening for active agents that are suitable for skin care, and more particularly useful for preventing and/or treating acneic and/or oily or acne-prone and/or oily skin.

The aim of the present invention is to satisfy these needs.

According to one of its aspects, the present invention relates to the use of an amino acid or nucleic acid sequence of the invention, for screening for or in a process for screening for, in particular in vitro or ex vivo, active agents that are particularly suitable for skin care.

The screened active agents may particularly be suitable for caring for acne-prone and/or oily skin, and/or for the prevention and/or treatment of acne, of oily skin and of imperfections.

A use or a process of the invention may comprise the comparison of a measurement of the activity, expression or maturation of an amino acid sequence or of a nucleic acid sequence in accordance with the invention, with a reference measurement.

A reference measurement may be as previously defined.

In particular, a reference measurement may be a quantitative or qualitative value relating to the expression, maturation or activity of said sequences, determined in a sample in the absence of active agent tested.

Thus, a reference measurement may be obtained by repeating the steps of a process of the invention, and in particular steps a), b) and c) of a process of the invention as defined previously, in the absence of biological or chemical compounds to be tested.

A comparison of the test measurement with a reference measurement, and an observation of a deviation or an absence of deviation between the two measurements makes it possible to draw information regarding the effect of the active agent tested.

The quantitative or qualitative determination of the expression, maturation or activity of an amino acid sequence or of a nucleic acid sequence of the invention may be performed via any method known to those skilled in the art, and in particular as previously described.

According to one embodiment, the screening of an active agent that is capable of modulating the activity of an amino acid sequence of the invention may be performed by measuring the activity or the expression of a target molecule belonging to the signalling or metabolic pathways in which said amino acid sequence may be involved, for instance a reporter gene system.

According to one embodiment, a process of the invention may be performed in an acellular system, i.e. in a system which does not comprise cells but which reproduces cell functions, or in an isolated cell sample.

A process in accordance with the invention can be carried out on an isolated cell sample, on an acellular sample, on an isolated amino acid sequence or on an isolated nucleic acid sequence of the invention, obtained by skin biopsy, from cells in culture, in particular from an epidermal model, or from a non-invasive skin surface sample, in particular taken by tape-stripping of the stratum corneum or by simple washing, as previously described.

Advantageously, by way of a cell sample that is suitable for the invention, mention may be made of a sample of keratinocytes or any other skin cell type expressing an amino acid sequence of the invention.

Preferably, the screening for an active agent may be performed by measuring the variation of the expression, in the presence and in the absence of the screened active agent, of an amino acid sequence of the invention, and preferably represented by a sequence chosen from SEQ ID NO: 16, SEQ ID NO: 17, or SEQ ID NO: 18, or an analog or fragment thereof.

The active agents resulting from a screening according to the invention can be advantageously used for cosmetic or therapeutic purposes, in particular for caring for acne-prone and/or oily skin or for the treatment of acneic and/or oily skin.

According to one embodiment, an active agent of the invention may be an agent that stimulates the activity, expression or maturation of an amino acid sequence of the invention.

By way of modulating agent that can be screened for according to a use or a process in accordance with the invention, mention may also be made of antibodies or interfering RNAs.

For the purposes of the present invention, the term “one” should be understood, unless otherwise indicated, as meaning “at least one”.

The examples and figures below are presented as non-limiting illustrations of the invention.

### EXAMPLES

### Example 1

### Determination of the Inhibitory Activity of Dermcidin on P. acnes Growth

A micromethod was developed in a microplate.

The test principle is based on bringing decreasing concentrations of antimicrobial peptide (DCD-1L) into contact with an identical inoculum of P. acnes in a culture medium conducive to the growth of P. acnes. After incubation of the microplate for 48 h with the P. acnes strain, the optical density of the sample (620 nm) is measured and the results are expressed as percentage growth calculated relative to a growth control without antimicrobial peptide (negative control).

In addition, the inoculum is also brought into contact with triclosan in order to have a positive control.

The minimum inhibitory concentration or MIC is the lowest concentration of antibiotic that is sufficient to inhibit, in the laboratory, i.e. in vitro, the growth of a bacterial strain (bacterial colony).

The concentration range chosen ranged from 5 to 100 μg/ml.

Each concentration was tested in triplicate.

The inhibitory activity of the peptides was evaluated on the following microorganism:
P. acnes ATCC 6919 (Gram+bacterium)

| | | MIC in |
|---|---|---|
| Name of the | Supplier Info | μg/ml |
| antimicrobial peptide | Batch reference | P. acnes |
| Recombinant human | Interchim (Serotec) | 75 |
| HBD2 | product code | |
| | PHP161X, batch | |
| | 221210 | |
| RNASE 7 | Human Plv extraction | >100 |
| | (heel horn) | |
| Recombinant human | Interchim (Protera) | >100 |
| S100A7 | folding verified by | |
| | NMR batch EN00510- | |
| | 23/24/25 | |
| Lacritin | Synthesized by | 100 |
| | hybrogenics Ref: Hgx | |
| | 2238v1 | |
| Dermcidin (DCD1L) | Synthesized by jpt | <5 |
| SEQ ID NO: 20 | | |

It is observed that the growth of P. acnes is greatly inhibited in the presence of a low concentration of the DCD1L peptide.

### Example 2

### Cosmetic Composition

A cosmetic composition comprising the following ingredients (% by weight) is prepared:

| | |
|---|---|
| Dermcidin | 1 |
| antioxidant | 0.05 |
| isopropanol | 40.0 |
| preservative | 0.30 |
| water | qs 100% |

### Example 3

### Pharmaceutical Composition

A therapeutic composition comprising the following ingredients (% by weight) is prepared:

| | |
|---|---|
| Dermcidin | 5.00 |
| Isopropanol | 40.00 |
| Preservative | 0.30 |
| Water | qs 100% |

### Example 4

### Facial Care Cream

A cosmetic composition comprising the following ingredients (% by weight) is prepared:

| | |
|---|---|
| Arachidyl behenyl alcohol/arachidyl glusoside | 2.0 |
| Isohexadecane | 7.0 |
| Dermcidin | 5.00 |
| Glycerol | 2.0 |
| Water | qs 100% |

### LITERATURE

Anderson, R. K. and W. L. Kenney (1987). “Effect of age on heat-activated sweat gland density and flow during exercise in dry heat” J. Appl. Physiol. 63(3): 1089-1094.
Baechle, D., T. Flad, et al. (2006). “Cathepsin D is present in human eccrine sweat and involved in the postsecretory processing of the antimicrobial peptide DCD-1L” J. Biol. Chem. 281(9):5406-5415.
Cunningham, T. J., L. Hodge et al. (1998) “Identification of a survival-promoting peptide in medium conditioned by oxidatively stressed cell lines of nervous system origin” J. Neurosci 18(18):7047-7060.
Flad, T., R. Bogumil et al. (2002) “Detection of dermcidin-derived peptides in sweat by ProteinChip Technology.” J. Immunol. Methods 270(1): 53-62.
Kenney, W. L. and S. R. Fowler (1988) “Methylcholine-activated eccrine sweat gland density and output as a function of age” J. Appl. Physiol. 65(3):1082-1086.
Kyte et al., (1982), J. Mol. Biol., 157: 105.
Mehul B. et al., (2000) “Identification and Cloning of a New Calmodulin-like Protein from Human Epidermis” J. Biol. Chem. 275(17):12841-12847
Moreira, D. F, B. E. Strauss et al. (2008) “Genes up- and down-regulated by dermcidin in breast cancer: a microarray analysis” Genet. Mol. Res. 7(3): 925-932.
Niyonsaba, F. A. Suzuki et al. (2009) “The human antimicrobial peptide dermcidin activates normal human keratinocytes” Br. J. Dermatol. 160(2): 243-249.
Rieg, S. C., Garbe et al. (2004) “Dermcidin is constitutively produced by eccrine sweat gland and is not induced in epidermal cells under inflammatory skin conditions” Br. J. Dermatol. 151(3): 534-539.
Rieg S., II Steffen et al. (2005) “Deficiency of dermcidin-derived antimicrobial peptides in sweat of patients with atopic dermatitis correlates with an impaired innate defense of human skin in vivo” J. Immunol. 174(12):8003-8010.
Sakurada K., T. Akutsu, et al., (2009) “Detection of dermcidin for sweat identification by real-time RT-PCR and ELISA” Forensic. Sci. Int. 2010 Jan. 30; 194(1-3):80-4. Epub 2009 Nov. 13.
Sambrook et al., 1989, Vol. I-III, Coldspring Harbor Laboratory, Coldspring Harbor Press, N.Y.
Schittek, B, R. Hipfel et al. (2001): “Dermcidin: a novel human antibiotic peptide secreted by sweat glands” Nat. Immunol. 2(12): 1133-11137.
Watchorn, T. M. N. Dowidar et al. (2005) “The chachectic mediator proteolysis inducing factor activates NF-kappaB and STAT3 in human Kupffer cells and monocytes” Int. J. Oncol. 27(4): 1105-1111.
Wiese et al., (2007) “Protein labeling by iTRAQ: A new tool for quantitative mass spectrometry in proteome research” Proteomics 7(3):340-350
Zieske et al. (2006) “A perspective on the use of iTRAQ reagent technology for protein complex and profiling studies” J. Exp. Bot. 57(7):1051-1058.

1. A process for preventing the appearance of imperfections and/or treating and/or acne-prone and/or oily skin in an individual who is in need thereof, which comprises administering, to said individual, at least one amino acid sequence or of a composition containing same, chosen from SEQ ID NO: 11 to SEQ ID NO: 20, or an analog or fragment thereof.

2. A process for treating or preventing acneic and/or oily skin or preventing the appearance of imperfections in an individual who is in need thereof, which comprises administering, to said individual (i) at least one amino acid sequence or of a composition containing same, chosen from SEQ ID NO: 11 to SEQ ID NO: 20, or an analog or fragment thereof, or (ii) at least one nucleic acid sequence chosen from SEQ ID NO: 1 to SEQ ID NO: 10, or (iii) at least one agent for modulating the activity, expression or maturation of said amino acid sequence or of said nucleic acid sequence.

3. The process as claimed in claim 1, wherein said amino acid sequence chosen from SEQ ID NO: 11 to SEQ ID NO: 20, or an analog or fragment thereof, is chosen from SEQ ID NO: 16, SEQ ID NO: 17, SEQ ID NO: 18, SEQ ID NO: 19, SEQ ID NO: 20, or an analog or fragment thereof.

4. The process as claimed in claim 1, wherein said amino acid sequence chosen from SEQ ID NO: 16, SEQ ID NO: 17, SEQ ID NO: 18, SEQ ID NO: 19, SEQ ID NO: 20, or an analog or fragment thereof, is SEQ ID NO: 20, or an analog or fragment thereof.

5. The process as claimed in claim 2, wherein said nucleic acid sequence is chosen from SEQ ID NO: 6, SEQ ID NO: 7, SEQ ID NO: 8, SEQ ID NO: 9, SEQ ID NO: 10.

6. The process as claimed in claim 2, wherein said nucleic acid sequence is SEQ ID NO: 10, or an analog or fragment thereof.

7. The process as claimed in claim 1, wherein said at least one amino acid sequence represents from 0.00001% to 5% of the total weight of the composition.

8. A process for preventing the appearance of imperfections and/or preventing and/or treating acneic and/or oily and/or acne-prone and/or oily skin in an individual who is in need thereof, comprising administering, to said individual, at least one composition comprising, as active agent, (i) at least one amino acid sequence chosen from SEQ ID NO: 11 to SEQ ID NO: 20, or an analog or fragment thereof, or (ii) at least one nucleic acid sequence as defined in claim 2, or (iii) at least one agent for modulating the activity, the expression or the maturation of said sequences.

9. The process as claimed in claim 1 wherein said composition is a cosmetic or pharmaceutical.

10. (canceled)

11. An in vitro or ex vivo process for characterizing an acneic and/or oily condition of the skin of a subject, comprising the steps consisting in:
a) performing, in an isolated sample of skin of a subject, a qualitative or quantitative measurement of the expression, maturation or activity of (i) of at least one amino acid sequence chosen from SEQ ID NO: 11 to SEQ ID NO: 20, or an analog or fragment thereof, or of at least one nucleic acid sequence chosen from SEQ ID NO: 1 to SEQ ID NO: 10 or an analog or fragment thereof as a biomarker, and
b) comparing said measurement performed in step a) with a reference measurement,
c) concluding that a reduction in the activity, expression or maturation of said biomarker relative to said reference measurement is indicative of acneic and/or oily or acne-prone and/or oily skin in the subject.

12. (canceled)

13. A process for screening for, in vitro or ex vivo, active agents that are capable of treating and/or preventing acneic and/or oily and/or acne-prone and/or oily skin and/or for preventing the appearance of imperfections, or modulating the activity, expression or maturation of an amino acid sequence chosen from SEQ ID NO: 11 to SEQ ID NO: 20, or an analog or fragment thereof, or of a nucleic acid sequence an amino acid sequence chosen from SEQ ID NO: 11 to SEQ ID NO: 20, or an analog or fragment thereof, or a nucleic acid sequence chosen from SEQ ID NO: 1 to SEQ ID NO: 10, comprising at least the steps consisting in:
a) placing said amino acid sequence or said nucleic acid sequence under conditions favorable to the activity, expression or maturation of said sequences,
b) bringing said amino acid sequence or said nucleic acid sequence into contact with at least one active agent to be tested,
c) performing a qualitative or quantitative measurement of the expression, maturation or activity of said amino acid sequence or of said nucleic acid sequence, and
d) comparing said measurement with a reference measurement,
e) concluding that the increase in the activity, expression or maturation of an amino acid sequence in accordance with the invention is indicative of the efficacy of said active agent for preventing or treating acne-prone or acneic skin or preventing the appearance of imperfections.

14. The process as claimed in claim 2, wherein said amino acid sequence is chosen from SEQ ID NO: 16, SEQ ID NO: 17, SEQ ID NO: 18, SEQ ID NO: 19, SEQ ID NO: 20, or an analog or fragment thereof.

15. The process as claimed in claim 2, wherein said amino acid sequence is SEQ ID NO: 20, or an analog or fragment thereof.

16. The process as claimed in claim 2, wherein said at least one amino acid sequence represents from 0.00001% to 5% of the total weight of the composition.

17. The process as claimed in claim 1, wherein said at least one amino acid sequence represents from 0.001% to 5% of the total weight of the composition.

18. The process as claimed in claim 2, wherein said at least one amino acid sequence represents from 0.001% to 5% of the total weight of the composition.

19. The process as claimed in claim 1, wherein said at least one amino acid sequence represents from 0.001% to 1% of the total weight of the composition.

20. The process as claimed in claim 2, wherein said at least one amino acid sequence represents from 0.001% to 1% of the total weight of the composition.

21. The process as claimed in claim 3, wherein said at least one amino acid sequence represents from 0.00001% to 5% of the total weight of the composition.

22. The process as claimed in claim 4, wherein said at least one amino acid sequence represents from 0.00001% to 5% of the total weight of the composition.
